(19) United States
(12) Patent Application Publication
(10) Pub. No.: US 2010/0010521 A1
(43) Pub. Date: Jan. 14, 2010

(54) CUTTING BALLOON WITH MOVABLE MEMBER

(75) Inventor: Michael R. Kurrus, Ellettsville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/171,003

(22) Filed: Jul. 10, 2008

(51) Int. Cl. A61B 17/22

(52) U.S. Cl. 606/159

(57) ABSTRACT

A balloon catheter and method of use of the balloon catheter are described that may be used to dilate hardened regions of a stenosed region within a body vessel. The balloon catheter is provided with at least one wire that extends between a distal member and a proximal member. The distal member is fixedly attached to the catheter shaft, and the proximal member is slidably disposed along the outer diameter of the shaft. Inflation of the balloon causes the proximal member and the proximal end of the wire attached thereto to distally move along the shaft. Movement of the wire in the distal direction enables subsequent angioplasty to be performed. Deflation of the balloon causes the proximal member and the proximal end of the wire to proximally move along the shaft after completion of the angioplasty procedure.

### BACKGROUND

The present invention relates generally to medical devices and more particularly to balloon catheters used to dilate narrowed portions of a lumen.

Balloon catheters are widely used in the medical profession for various intraluminal procedures. One common procedure involving the use of a balloon catheter relates to angioplasty dilation of coronary or other arteries suffering from stenosis (i.e., a narrowing of the arterial lumen that restricts blood flow).

Although balloon catheters are used in many other procedures as well, coronary angioplasty using a balloon catheter has drawn particular attention from the medical community because of the growing number of people suffering from heart problems associated with stenosis. This has lead to an increased demand for medical procedures to treat such problems. The widespread frequency of heart problems may be due to a number of societal changes, including the tendency of people to exercise less while eating greater quantities of unhealthy foods, in conjunction with the fact that people generally now have longer life spans than previous generations. Angioplasty procedures have become a popular alternative for treating coronary stenosis because angioplasty procedures are considerably less invasive than other alternatives. For example, stenosis of the coronary arteries has traditionally been treated with bypass surgery. In general, bypass surgery involves splitting the chest bone to open the chest cavity and grafting a replacement vessel onto the heart to bypass the blocked, or stenosed, artery. However, coronary bypass surgery is a very invasive procedure that is risky and requires a long recovery time for the patient.

To address the increased need for coronary artery treatments, the medical community has turned to angioplasty procedures, in combination with stenting procedures, to avoid the problems associated with traditional bypass surgery. Typically, angioplasty procedures are performed using a balloon-tipped catheter that may or may not have a stent mounted on the balloon (also referred to as a stented catheter). The physician performs the angioplasty procedure by introducing the balloon catheter into a peripheral artery (commonly one of the leg arteries) and threading the catheter to the narrowed part of the coronary artery to be treated. During this stage, the balloon is uninflated and collapsed onto the shaft of the catheter in order to present a low profile which may be passed through the arterial lumens. Once the balloon is positioned at the narrowed part of the artery, the balloon is expanded by pumping a mixture of saline and contrast solution through the catheter to the balloon. As a result, the balloon presses against the inner wall of the artery to dilate it. If a stent is mounted on the balloon, the balloon inflation also serves to expand the stent and implant it within the artery. After the artery is dilated, the balloon is deflated so that it once again collapses onto the shaft of the catheter. The balloon-tipped catheter is then retracted from the arteries. If a stent is mounted on the balloon of the catheter, the stent is left permanently implanted in its expanded state at the desired location in the artery to provide a support structure that prevents the artery from collapsing back to its pre-dilated condition. On the other hand, if the balloon catheter is not adapted for delivery of a stent, either a balloon-expandable stent or a self-expandable stent may be implanted in the dilated region in a follow-up procedure. Although the treatment of stenosed coronary arteries is one common example where balloon catheters have been used, this is only one example of how balloon catheters may be used and many other uses are also possible.

One problem that may be encountered with conventional angioplasty techniques is the proper dilation of stenosed regions that are hardened and/or have become calcified. Stenosed regions may become hardened for a variety of reasons, such as the buildup of atherosclerotic plaque or other substances. Hardened regions of stenosis can be difficult to completely dilate using conventional balloons because hardened regions tend to resist the expansion pressures applied by conventional balloon catheters. Furthermore, the stenosed regions may become fully occluded to the extent that the entire lumen of the vessel is blocked, thereby preventing a dilation device from being deployed within the stenosed region. Although the inventions described below may be useful in treating hardened regions of stenosis, the claimed inventions may also solve other problems as well.

### SUMMARY

The invention may include any of the following aspects in various combinations and may also include any other aspect described below in the written description or in the attached drawings.

A balloon catheter for dilation of a lumen, comprising: a balloon having a distal portion, and a proximal portion, wherein at least a length of an outer surface of the balloon is expandable; a shaft having a longitudinal axis, a distal portion and a proximal portion, the balloon being mounted on the distal portion of the shaft, wherein the shaft further comprises an inflation lumen extending therethrough in fluid communication with an interior region of the balloon, the balloon thereby being expandable between a deflated state and an inflated state; at least one wire extending along the balloon, the at least one wire comprising a distal end attached to the distal portion of the shaft at a location distally of the balloon, the at least one wire further comprising a proximal end affixed to a proximal movable member, the proximal movable member slidably disposed over the shaft at a location proximally of the balloon, whereby inflation of the balloon causes the movable member and the proximal end of the wire to distally move along the shaft.

A balloon catheter for dilation of a lumen, comprising: a balloon having a distal portion, and a proximal portion, wherein at least a length of an outer surface of the balloon is expandable; a shaft having a longitudinal axis, a distal portion and a proximal portion, the balloon being mounted on the distal portion of the shaft, wherein the shaft further comprises an inflation lumen extending therethrough in fluid communication with an interior region of the balloon, the balloon thereby being expandable between a deflated state and an inflated state; and at least one wire extending between a proximal movable member located proximal of the balloon and a fixed distal member located distal of the balloon, the wire configurable between a first state and a second state, the wire in the first state substantially straight and the wire in the second state expanded outward.

A balloon catheter for dilation of a lumen, comprising: a balloon having a distal portion, and a proximal portion, wherein at least a length of an outer surface of the balloon is expandable; a shaft having a longitudinal axis, a distal portion and a proximal portion, the balloon being mounted on the distal portion of the shaft, wherein the shaft further comprises an inflation lumen extending therethrough in fluid communication with an interior region of the balloon, the balloon thereby being expandable between a deflated state and an inflated state; and a wire extending along the outer surface of the balloon, the wire comprising a sharpened region between a first unsharpened region and a second unsharpened region, the sharpened region being offset from a working diameter of the balloon when the balloon is in the deflated state and the sharpened portion being aligned with the working diameter when the balloon is in the inflated state.

### BRIEF DESCRIPTION OF THE DRAWINGS

The invention may be more fully understood by reading the following description in conjunction with the drawings, in which:

FIG. 1 is a side view of a balloon catheter including a cutting wire extending along a deflated balloon, the cutting wire extending between a movable member and a fixed member;

FIG. 2 shows the balloon catheter of FIG. 1 with the balloon inflated;

FIG. 3 shows a cross-sectional view of the balloon catheter of FIG. 2;

FIG. 4 shows a cross-sectional view of the sharpened section of the cutting wire;

FIG. 5 shows a partial cross-sectional view of the balloon catheter introduced into a stenosed region, showing the balloon deflated and the wires extending along the balloon between the collars;

FIG. 6 shows a partial cross-sectional view of the balloon catheter introduced into a stenosed region, showing the balloon inflated and the proximal ends of the wires advanced distally relative to FIG. 5 to enable the wires to extend along a working diameter of the inflated balloon;

FIG. 7 shows a partial cross-sectional view of the balloon catheter with the wires longitudinally extended compared to FIG. 6 after the stenosed region has been dilated;

FIG. 8 shows a cross-sectional view of a deflated balloon in a pleated configuration with cutting wires disposed between folds;

FIG. 9 shows a perspective view of the inflated balloon with two cutting wires extending therealong, each of the cutting wires having a sharpened section along the working diameter and a non-sharpened section along the proximal and distal neck regions of the balloon; and

FIG. 10 shows the balloon catheter with a movable member connected to the proximal ends of the wires, the movable member assisting with the movement of the proximal ends of the wires.

### DETAILED DESCRIPTION

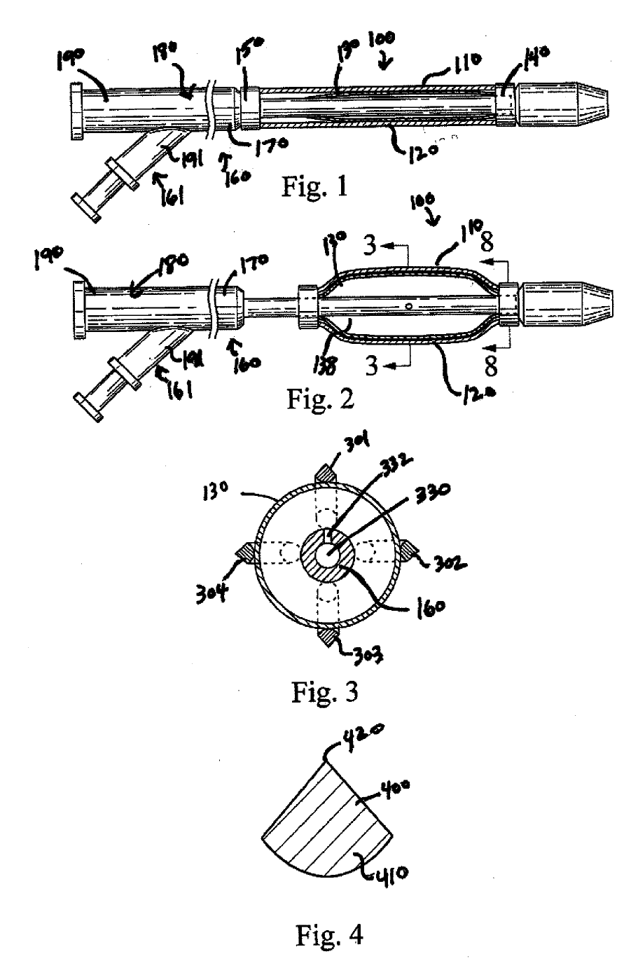

FIGS. 1-2 show a balloon catheter 100 used for dilating a body lumen. FIG. 1 shows the balloon catheter 100 having cutting wires 110 and 120 longitudinally extending along a deflated balloon 130. FIG. 2 shows the balloon catheter 100 with the proximal ends of the cutting wires 110 and 120 advanced distally relative to the proximal ends in FIG. 1. This distal advancement of the proximal ends of the cutting wires 110 and 120 enables the wires 110 and 120 to extend along the working diameter and neck portions of the inflated balloon 130. Each of the wires 110 and 120 has a proximal end that is connected to a proximal movable member 150 and a distal end that is connected to a distal member 140. The operation of the balloon catheter 100 and a procedure for using the balloon catheter 100 will be described in further detail below.

The balloon catheter 100 includes a shaft 160 with a distal portion 170 and a proximal portion 180. The distal portion 170 of the catheter shaft 160 is designed to be introduced into a body lumen, such as a vessel. It also includes the region onto which the balloon 130 is disposed along, as shown in FIGS. 1 and 2. The proximal portion 180 (FIGS. 1 and 2) of the shaft 160 is designed to remain outside of the patient's body. A manifold 161 along the proximal portion 180 may be provided as shown in FIGS. 1 and 2. The manifold 161 may have a guidewire port 190 designed for receiving a guidewire and an inflation port 191 designed for receiving inflation fluid for inflating the balloon 130.

Still referring to FIG. 1, the distal and proximal ends of the wires 110 and 120 may be connected to the distal member 140 and proximal movable member 150 by any means known to one of ordinary skill in the art, including soldering or welding. The distal member 140 is fixedly attached to the shaft 160 as shown in FIGS. 1 and 2 so that it does not move during inflation and deflation of the balloon 130. A variety of structures are contemplated for distal member 140, including an annular connector which may be bonded to the outer surface of the shaft 160. Alternatively, the distal ends of the wires 110 and 120 may be directly connected to the shaft 160 at a location distal to the balloon 130.

The proximal movable member 150 is slidably disposed along the shaft 160. A variety of structures are contemplated for proximal movable member 150. Preferably, the proximal movable member 150 is an annular collar having an inner diameter which is slightly larger than the outer diameter of the shaft 160. The annular collar is designed to slidably move along the outer surface of the shaft 160 in the distal and proximal directions as the balloon 130 inflates and deflates, respectively. The proximal end of each of the wires 110 and 120 may be connected to the proximal movable member 150 by soldering, welding, or other means known in the art. Movement of the proximal movable member 150 allows the wires 110 and 120 to be pushed during inflation of the balloon 130 and pulled during deflation of the balloon 130. As a result, movement of the proximal movable member 150 provides the necessary degree of freedom for the wires 110 and 120 to longitudinally move during inflation and deflation of the balloon 130.

The orientation of the wires 110 and 120 may be dependent upon the separation between the distal member 140 and the proximal movable member 150. FIG. 1 shows the preferred configuration of the balloon 130 during delivery to a body lumen and subsequent withdrawal from the body lumen. Specifically, the proximal member 150 is preferably pushed back by the spring force of the wires 110 and 120 which may have a tendency to return to their relaxed, straightened position, as shape memory alloys or traditional metals not deformed beyond their yield strength behave. Accordingly, the proximal member 150 is shown returning to its proximal most location along the shaft 160 so that the wires 110 and 120 are configured in substantially close proximity to the outer surface of the shaft 160 to create a low profile of the balloon catheter 100 during delivery and withdrawal from the body lumen. The proximal ends of the wires 110 and 120 extend beyond the proximal end of the balloon 130 in its deflated state (FIG. 1). Such an orientation of the wires 110 and 120 may significantly reduce the risk of the wires 110 and 120 from possessing sufficient slack to be disposed away from the shaft 160 and inadvertently traumatize tissue during delivery and withdrawal of the balloon catheter 100.

FIG. 2 shows the balloon catheter 100 with the balloon 130 in its inflated state. As the balloon 130 expands in diameter to its inflated state, the distance between the proximal movable member 150 and the distal member 140 shortens as the wires 110 and 120 expand with the balloon 130. The distal member 140 remains stationary thereby causing each of the proximal ends of the wires 110 and 120 to pull on the proximal movable member 150. The pulling force exerted on the proximal movable member 150 causes the movable member 150 and the proximal ends of the wires 110 and 120 attached thereto to move along the shaft 160 in the distal direction towards the proximal tapered portion (i.e., the neck) of the inflated balloon 130 (FIG. 2). When the balloon 130 is deflated to the deflated state of FIG. 1, the wires 110 and 120 exert a pushing force against movable member 150 to proximally move the member 150 to the location shown in FIG. 1. This feature allows inflation of the balloon 130 to its expanded state without tearing the otherwise rigid wires 110 and 120 from the surface of the balloon 130, which is a problem typically encountered in the cutting balloon art.

Referring to FIG. 9, the wires 110 and 120 may be sharpened in selective areas. The sharpened region of the wires 110 and 120 may extend only along the working diameter of the balloon 130. The portions of the wires 110 and 120 extending along the proximal and distal neck regions 910 and 920 of the balloon 130 may comprise non-sharpened regions. Such a design takes into account that dilation of a body lumen is typically performed along the working diameter of the balloon 130 rather than the proximal and distal neck regions 910 and 920 of the balloon 130. The longitudinal length of the working diameter may be defined as the distance between the balloon 130 proximal end 131, where the tapered proximal neck region 910 meets the working diameter, and the balloon distal end 132, where the distal neck region 920 meets the working diameter.

FIGS. 1 and 2 further illustrate the sharpened and nonsharpened regions along the deflated and inflated balloon 130. FIG. 1 shows that each of the wires 110 and 120 comprises a sharpened region 191 that is disposed between a first unsharpened region 192 and a second unsharpened region 193. The sharpened region 191 is offset from the working diameter of the deflated balloon 130. FIG. 2 shows that when the balloon 130 inflates, the sharpened region 191 becomes aligned with the working diameter of the balloon 130. The unsharpened region 230 becomes aligned with the proximal neck of the inflated balloon 130 and the unsharpened region 220 becomes aligned with the distal neck of the inflated balloon 130.

A movable handle 192 may also be provided as shown in FIG. 10. The movable handle 192 is slidable along the proximal region of shaft 160, as indicated by the arrow in FIG. 10. There may be circumstances when deflation of the balloon 130 to its deflated state does not automatically cause the wires 110 and 120 to sufficiently push on movable member 150 so as to proximally retract the movable member along with wires 110 and 120 into the extended configuration shown in FIG. 1. In such a scenario, the movable handle 192 may assist in moving the proximal end of the wires 110 and 120 in the proximal direction during deflation of the balloon 130. There may also be circumstances when inflation of the balloon 130 to its expanded state does not automatically cause the proximal ends of the wires 110 and 120 to distally move towards the proximal neck 910 (FIG. 9) of inflated balloon 130. Accordingly, the movable handle 192 may also assist in moving the proximal end of the wires 110 and 120 in the distal direction during inflation of the balloon 130 to an expanded state.

Lumens may be provided within the shaft 160 as passageways for connecting the proximal ends of corresponding wires 110 and 120 to the movable handle 192. Alternatively, a separate wire or member 198 may be affixed to the movable member 150. The separate wire or member 198 may then connect to the handle 192 via a lumen 194 (FIG. 10) within the shaft 160. The separate wire or member 198 is relatively more rigid than the wires 110 and 120 and therefore may be more effective than wires 110 and 120 for transmitting compressive and tensile forces from the handle 192 to the wires 110 and 120 during inflation and deflation of the balloon 130.

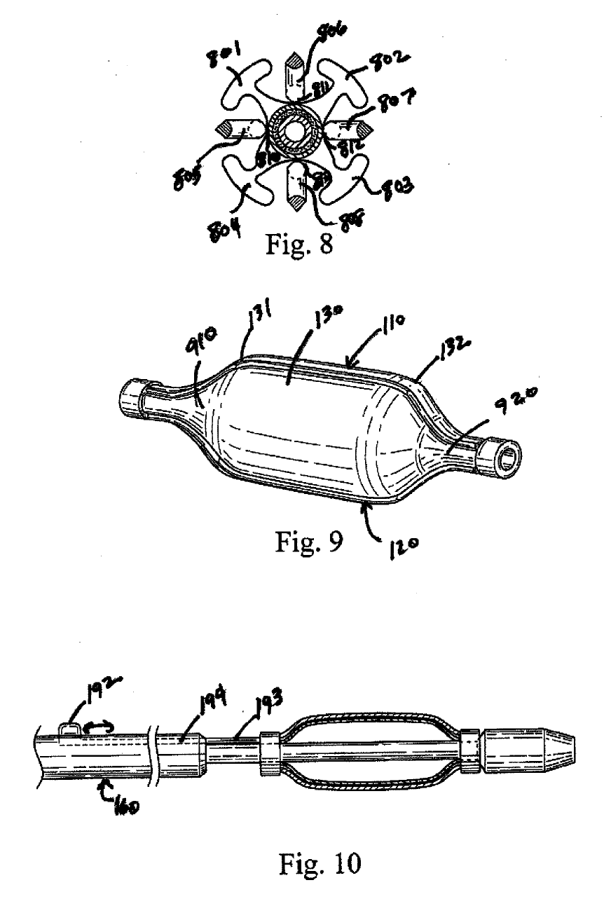

Referring to FIGS. 1 and 2, shaft 160 is shown to have a smaller diameter along the region in which balloon 130 is disposed. The smaller diameter of shaft 160 may enable a lower profile of the balloon catheter 100 during navigation of the balloon catheter 100. This may be particularly useful if the folds 801-804 of the balloon 130 (FIG. 8) inadvertently open up during navigation of the balloon catheter 100.

FIG. 8 shows a cross-sectional view of a deflated balloon 130 in a pleated configuration with four cutting wires 805-808 disposed between respective folds 801-804 of the balloon 130. Such a pleated configuration in which the folds 801-804 shield sharp edges of the wires 805-808 help to avoid unnecessary trauma to tissue during delivery and withdrawal of the balloon catheter 100. FIG. 8 also shows that the ends of the wires 805-808 are connected to the proximal and/or distal member by soldering or welding at locations 810-813.

As shown in FIG. 3, the shaft 160 may have a guidewire lumen 330 and an inflation lumen 332. Four wires 301-304 are shown disposed about the balloon 130. The guidewire lumen 330 is in fluid communication with the guidewire port 190 (FIG. 1). Typically, the guidewire lumen 330 extends longitudinally through the shaft 160 to the distal end of the shaft 160. Thus, the guidewire lumen 330 may be used to thread the balloon catheter 100 through narrow, tortuous vessels in a manner well known to those of ordinary skill in the art. The inflation lumen 332 is in fluid communication with the interior region 138 of the balloon 130. Thus, the balloon 130 may be inflated by supplying a pressurized fluid, such as saline, to the inflation port 191 (FIG. 1). Similarly, the balloon 130 may be deflated from the inflated state by applying a negative pressure to the inflation port 191 (FIG. 1), which draws the fluid out of the balloon 130. The shaft 160 may also have one or more dilation wire lumens extending therethrough.

FIG. 4 shows a cross-sectional view of a cutting wire 400. Numerous cross-sectional shapes of the cutting wire 400 are contemplated. FIG. 4 shows an embodiment in which the cutting wire has a tear drop cross-sectional shape. The bottom rounded portion 410 would preferably be disposed along the outer surface of the balloon 130. Such a rounded atraumatic surface of the wire 410 may avoid unnecessarily perforating the surface of the balloon 130. The sharpened edge 420 of the wire 400 is preferably oriented away from the balloon catheter 100 and towards the stenosed region. Numerous means for forming a tear-dropped cross-sectional wire 400 are contemplated. In one example, the raw material for the cutting wire 410 may be a circular cross-sectional wire. The circular cross-sectional wire may then be cut with a grinder at a predetermined angle to create the sharpened edges 420.

The wires 110 and 120 may be formed from metallic, plastic or other suitable biocompatible materials. Traditional metals, such as stainless steel, may be used as long as the balloon 130 does not bend the wires 110 and 120 beyond their yield strength. Preferably, the wires 110 and 120 are formed from a superelastic alloy, such as nitinol. The nitinol may be imparted with shape memory characteristics as known in the art to create a linear shape. In other words, the wires 110 and 120 will revert to the linear shape in their relaxed state when stress from the wires 110 and 120 is removed. Accordingly, during deflation of the balloon 130, the wires 110 and 120 will tend to return to their linear shape as the balloon 130 is deflated, thereby compressing the outer surface of the balloon 130 and facilitating deflation of the balloon 130

Although the balloon catheter 100 has been described with the distal member 140 fixed and the proximal movable member 150 movable along the distal region 170 of the shaft 160, an alternative design may be utilized in which the proximal member 150 remains fixed and the distal member 140 is allowed to slide back and forth along the distal region 170 of the shaft 160 during inflation and deflation of the balloon 130.

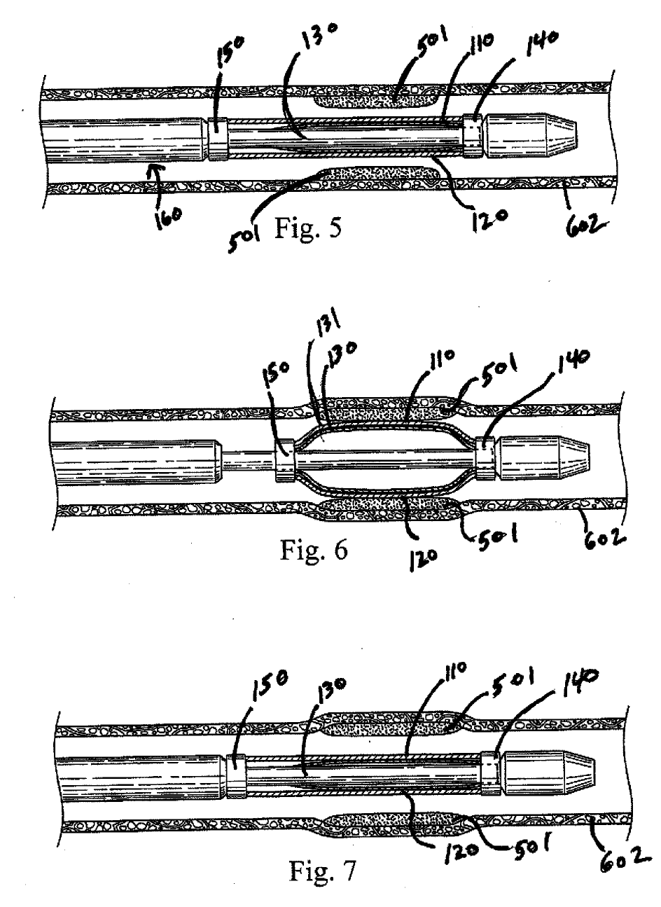

One preferred method for using the balloon catheter 100 is shown in FIGS. 5-7. As shown in FIG. 5, the balloon catheter 100 may be threaded to the stenosed region 501 with the wires 110 and 120 extending along the balloon 130 in their retracted state between the proximal movable member 150 and the distal member 140. The proximal member 150 may be sufficiently pushed back by the spring force of the wires 110 and 120 which have a tendency to return to their relaxed, straightened position. This may cause the proximal end of the wires 110 and 120 to proximally extend past the proximal end of the deflated balloon 130, as shown in FIG. 5. This may enable positioning of the wires 110 and 120 substantially against the surface of the shaft 160 to create a low profile of the balloon catheter 100 during delivery to the stenosed region 501 and thereby prevent the wires 110 and 120 from contacting or catching on the vessel wall 602 or other structures contained therein.

After navigating the balloon catheter 100 to the stenosed region 501, the shaft 160 may be positioned so that portions of the wires 110 and 120 overlying the working diameter of the balloon 130 are located adjacent the stenosed region 501. The wires 110 and 120 may be sharpened only along the working diameter of the balloon 130 (FIG. 9) since this is the region of the wires 110 and 120 which is typically involved in the dilation procedure.

After the shaft 160 and wires 110 and 120 are positioned within the stenosed region 501, inflation of the balloon 130 may occur. Because the distal ends of the wires 110 and 120 remain stationary at fixed distal member 140 during the inflation of the balloon 130 (FIG. 6) to its expanded state, the proximal ends of the wires 110 and 120 pull on proximal movable member 150. The member 150 is pulled in the distal direction towards the proximal neck 131 of the inflated balloon 130. The distance between the proximal movable member 150 and the distal member 140 decreases relative to the distance between the members 140 and 150 when the balloon 130 is in the deflated state as shown in FIG. 5. As the balloon 130 inflates to dilate the lumen of the vessel wall 602, the wires 110 and 120 are squeezed between the outer surface of the balloon 130 and the stenosed region 501. This is particularly beneficial for hardened or calcified stenosed regions 501 because the wires 110 and 120 may crack or breakup the hardened stenosis.

During the dilation procedure, optional handle 192 (FIG. 10) may be used if necessary to assist in slidable movement of the proximal movable member 150, thereby causing the proximal end of each of the wires 110 and 120 to advance in the distal direction towards the proximal neck region 910 (FIG. 9) of the balloon 130.

After the stenosed region has been dilated, the balloon 130 may be deflated. During deflation of the balloon 130, the diameter of the balloon 130 decreases such that the distance between the members 140 and 150 increases. Because the distal ends of the wires 110 and 120 remain stationary at fixed distal member 140 during the deflation of the balloon 130 (FIG. 7) to its deflated state, the proximal ends of the wires 110 and 120 push on proximal movable member 150 to slidably move the member 150 proximally as shown in FIG. 7. Handle 192 (FIG. 10) may be used if necessary to assist in proximal movement of the proximal member 150 and the proximal end of the wires 110 and 120. Preferably, the proximal movable member 150 is pushed to its proximal-most position, as shown in FIG. 7, to eliminate any slack in the wires 110 and 120 which may have a tendency to catch on vessel walls 602 or other strictures during withdrawal of the balloon catheter 100.

The structural design of balloon catheter 100 creates many advantages compared to other cutting balloons. For example, utilization of proximal and distal members 150 and 140 eliminates the commonly encountered difficulty of bonding wires directly to a balloon surface. Additionally, the wires 110 and 120 are always disposed along the outer surface of the balloon 130, thereby eliminating the additional steps of retracting and extending the wires 110 and 120, which may increase the time and difficulty of the procedure.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A balloon catheter for dilation of a lumen, comprising:
a balloon having a distal portion, and a proximal portion, wherein at least a length of an outer surface of the balloon is expandable;
a shaft having a longitudinal axis, a distal portion and a proximal portion, the balloon being mounted on the distal portion of the shaft, wherein the shaft further comprises an inflation lumen extending therethrough in fluid communication with an interior region of the balloon, the balloon thereby being expandable between a deflated state and an inflated state;
at least one wire extending along the balloon, the at least one wire comprising a distal end attached to the distal portion of the shaft at a location distally of the balloon, the at least one wire further comprising a proximal end affixed to a proximal movable member, the proximal movable member slidably disposed over the shaft at a location proximally of the balloon, whereby inflation of the balloon causes the movable member and the proximal end of the wire to distally move along the shaft.

2. The balloon catheter according to claim 1, further comprising a moveable handle to assist in moving the proximal end of the wire in the proximal direction during deflation of the balloon.

3. The balloon catheter according to claim 1, further comprising a moveable handle to assist in moving the proximal end of the wire in the distal direction during inflation of the balloon.

4. The balloon catheter according to claim 1, wherein the distal member comprises a fixed collar.

5. The balloon catheter of claim 1, wherein the deflated state comprises the wire being oriented substantially parallel to a longitudinal axis of the shaft, the wire proximally extending past the proximal end of the deflated balloon towards the movable member.

6. The balloon catheter of claim 1, wherein the distal member is disposed adjacent to the distal neck of the balloon and the proximal member is disposed away from the distal neck of the balloon when the balloon is deflated.

7. The balloon catheter of claim 1, wherein the wire is made of a superelastic alloy, the wire being formed to have a linear shape in a relaxed state.

8. The balloon catheter of claim 1, further comprising a plurality of wires, each of the plurality of wires having a proximal end and a distal end, wherein each of the proximal ends is connected to the proximal movable member and each of the distal ends is connected to the distal member.

9. The balloon catheter of claim 8, wherein each of the plurality of wires is longitudinally aligned with each other.

10. The balloon catheter according to claim 1, wherein the at least one wire comprises a tear-drop cross-sectional shape.

11. The balloon catheter according to claim 1, wherein the distal member is disposed adjacent to the distal neck of the balloon and the proximal member is disposed away from the distal neck of the balloon when the balloon is deflated and wherein the at least one wire comprises a tear-drop cross-sectional shape.

12. The balloon catheter according to claim 1, wherein the distal member comprises a fixed collar and wherein the deflated state comprises the wire being oriented substantially parallel to a longitudinal axis of the shaft, the wire proximally extending past the proximal end of the deflated balloon towards the movable member.

13. The balloon catheter according to claim 1, further comprising a plurality of wires, each of the plurality of wires having a proximal end and a distal end, wherein each of the proximal ends is connected to the proximal movable member and each of the distal ends is connected to the distal member and wherein each of the plurality of wires is longitudinally aligned with each other.

14. A balloon catheter for dilation of a lumen, comprising:
a balloon having a distal portion, and a proximal portion, wherein at least a length of an outer surface of the balloon is expandable;
a shaft having a longitudinal axis, a distal portion and a proximal portion, the balloon being mounted on the distal portion of the shaft, wherein the shaft further comprises an inflation lumen extending therethrough in fluid communication with an interior region of the balloon, the balloon thereby being expandable between a deflated state and an inflated state; and
at least one wire extending between a proximal movable member located proximal of the balloon and a fixed distal member located distal of the balloon, the wire configurable between a first state and a second state, the wire in the first state substantially straight and the wire in the second state expanded outward.

15. The balloon catheter according to claim 14, the balloon catheter comprising a plurality of wires circumferentially disposed relative to each other, and wherein the balloon has a plurality of creases about the outer surface of the balloon, the plurality of creases forming flaps when the balloon is in the deflated state, the flaps folding around each of the plurality of dilation wires.

16. The balloon catheter according to claim 15, wherein the flaps are substantially parallel with each other, the flaps being in alignment with the longitudinal axis of the balloon.

17. The balloon catheter according to claim 14, the wire further comprising a sharpened cross-section and a non-sharpened cross-section, wherein the wire in the second state is configured with the sharpened cross-section extending along the working diameter of the inflated balloon and the non-sharpened cross-section extending along a proximal neck and a distal neck of the inflated balloon.

18. A balloon catheter for dilation of a lumen, comprising:
a balloon having a distal portion, and a proximal portion, wherein at least a length of an outer surface of the balloon is expandable;
a shaft having a longitudinal axis, a distal portion and a proximal portion, the balloon being mounted on the distal portion of the shaft, wherein the shaft further comprises an inflation lumen extending therethrough in fluid communication with an interior region of the balloon, the balloon thereby being expandable between a deflated state and an inflated state; and
a wire extending along the outer surface of the balloon, the wire comprising a sharpened region between a first unsharpened region and a second unsharpened region, the sharpened region being offset from a working diameter of the balloon when the balloon is in the deflated state and the sharpened portion being aligned with the working diameter when the balloon is in the inflated state.

19. The balloon catheter of claim 18, wherein the first sharpened region is aligned with a proximal neck of the inflated balloon and the second sharpened region is aligned with a distal neck of the inflated balloon.

20. The balloon catheter of claim 18, wherein the wire comprises a proximal end attached to a proximal movable member located proximal of the balloon and a distal end attached to a fixed distal member located distally of the balloon.
